(19) United States
(12) Patent Application Publication
(10) Pub. No.: US 2025/0342945 A1
(43) Pub. Date: Nov. 6, 2025

(54) SYSTEM, METHOD, AND MOBILE APPLICATION FOR LOCATING MEDICAL CARE CENTERS

(71) Applicant: CARECOMPASS, Mary Esther, FL (US)

(72) Inventor: Edward Klugman, Mary Esther, FL (US)

(21) Appl. No.: 18/653,406

(22) Filed: May 2, 2024

(51) Int. Cl. G16H 40/20; G16H 10/60; G16H 40/67

(52) U.S. Cl. CPC G16H 40/20; G16H 10/60; G16H 40/67

(57) ABSTRACT

Systems, computer-implemented methods, and software applications for locating nearby medical care centers that provide medical care needed by a patient and for providing estimated wait times and directions to the medical care centers so the patient can select and easily navigate to a medical care center that best accommodates the patient's needs and schedule.

### BACKGROUND OF THE INVENTION

Patients needing medical care outside of a scheduled doctor's appointment often have multiple options of nearby urgent care centers, emergency rooms, pharmacies, and other medical care centers but no convenient way of determining which is the most appropriate for their medical needs or which has the capacity to attend to them quickly. The most common method for locating nearby medical care centers is to search for them via a web browser or mapping database. Such searching may identify nearby medical care centers but does not provide information about whether the medical care centers can provide the needed medical care or how long the patient is likely to wait to receive medical treatment.

### SUMMARY OF THE INVENTION

The present invention solves the above-described problems and similar problems and provides a distinct advance in the art of locating medical care centers. Embodiments of the present technology achieve these objectives and others by providing systems, computer-implemented methods, and software applications for locating nearby medical care centers that provide medical care needed by a patient and for providing estimated wait times and directions to the medical care centers so the patient can select and easily navigate to a medical care center that best accommodates the patient's needs and schedule.

A computer-implemented method in accordance with one embodiment of the invention can be at least partially implemented with a patient software application for execution on a mobile phone or other portable electronic device operated by a patient and a remote server that communicates with the portable electronic device via a communications network.

The patient software application can be used by the patent to identify nearby medical centers that are best suited for the patient's current medical needs and to obtain directions, driving times, and wait times for the medical care centers. The patient software application is configured to obtain patient location information from a GPS receiver on the patient's portable electronic device, receive from the patient a request for locations of nearby medical care centers, and transmit relevant data to the remote server. The patient software application may also receive patient information, such as the age and sex of the patient and the patient's current medical needs and medical history and provide this information to the remote server.

The remote server receives the data provided by the patient software application and analyzes it to determine locations of medical care centers near the patient. The remote server also analyzes data representative of estimated wait times for the medical care centers and determines a combined drive and wait time for each of the medical care centers. The remote server then transmits to the mobile electronic device information representative of the locations of the nearby medical care centers and combined drive and wait times so that the patient software application can display or otherwise provide such information to the patient.

In other embodiments, the remote server also analyzes the patient information and determines which of the nearby medical care centers are most relevant to the patient's current medical needs and transmits corresponding information to the portable electronic device so that the patient software application can display or otherwise provide such information to the patient.

This summary is provided to introduce a selection of concepts in a simplified form that are further described in the detailed description below. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF DRAWING FIGURES

Embodiments of the present invention are described in detail below with reference to the attached drawing figures, wherein:

FIG. 1 is a schematic diagram of computer and communications equipment that may implement embodiments of the present invention.

FIG. 2 is a schematic diagram of selected components of a portable electronic device that may be used to implement embodiments of the present invention.

FIG. 3 is a schematic diagram of selected components of a server computer system that may be used to implement embodiments of the present invention.

FIG. 4 is a flow diagram that represents computer-implemented methods of the invention and/or software and computer programs of the present invention.

FIG. 5 is an exemplary screen display that may be presented on a portable electronic device by the patient software application.

The drawing figures do not limit the present invention to the specific embodiments disclosed and described herein. The drawings are not necessarily to scale, emphasis instead being placed upon clearly illustrating the principles of the invention.

### DETAILED DESCRIPTION

Embodiments of the present technology provide systems, computer-implemented methods, and software applications for locating nearby medical care centers that provide medical care needed by a patient and for providing information about estimated wait times and directions to the medical care centers so the patient can select and easily navigate to a medical care center that best accommodates the patient's needs and schedule. As used herein, the term “medical care center” is meant to encompass urgent care centers, hospital emergency rooms, pharmacies with medical clinics, and all other type of medical care providers that operate without a scheduled appointment with a particular doctor.

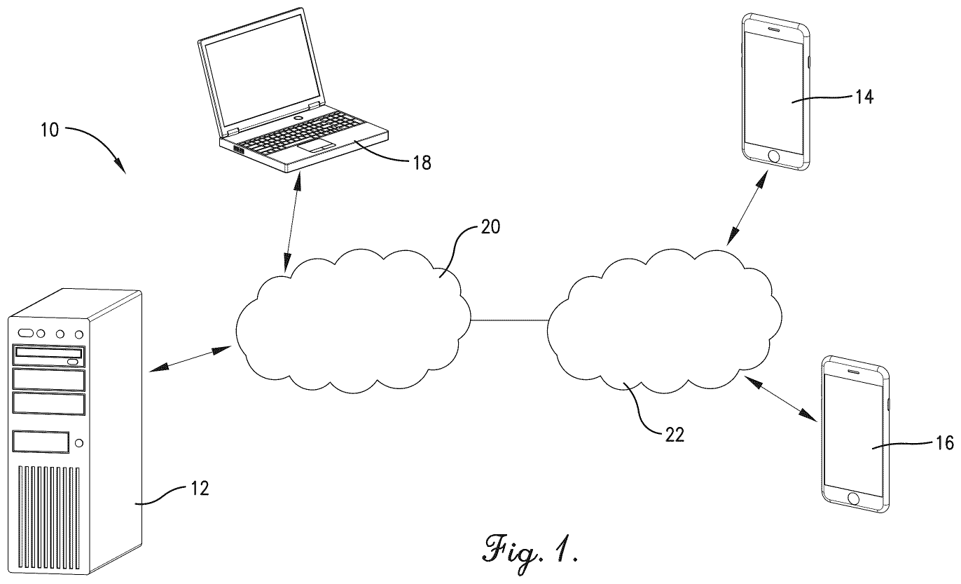

The invention is implemented with a computer and communications system and one or more computer programs for operating components of the system. An exemplary computer and communications system 10 is shown in FIG. 1 and broadly comprises a computer server system 12; one or more personal computing devices 14, 16, 18; and a communications network 20.

The computer server system 12, also referred to herein as a “remote server”, receives, stores, and provides access to the data and information described herein and implements one or more computer programs for performing at least some of the functions described herein. The computer server system 12 also provides a web-based portal that can be accessed by the personal computing devices 14, 16, 18 and other devices.

Embodiments of the computer server system 12 may include one or more servers running any operating software systems and the computer programs described herein. As shown in FIG. 3, the computer server system 12 includes a processor 24 or processor element, memory elements 26, and a communications element 28. The server computer also includes other hardware, firmware, and software and may have access to processing elements, computer memory, and other hardware and software on other computers for receiving, storing, accessing, and transmitting data and information as described herein. The computer server system 12 also includes or has access to conventional web hosting operating software, searching algorithms, and an Internet connection and is assigned a URL and corresponding domain name so that it can be accessed via the Internet or any other communications network in a conventional manner.

The processor 24, also referred to herein as a “processing element”, may include any number of processors, microprocessors, microcontrollers, DSPs, field-programmable gate arrays (FPGAs), analog and/or digital application-specific integrated circuits (ASICs), or the like, or combinations thereof. The processing element 24 may generally execute, process, or run instructions, code, code segments, software, firmware, programs, applications, apps, processes, services, daemons, or the like. The processing element may also include hardware components, such as finite-state machines, sequential and combinational logic, and other electronic circuits that may perform the functions necessary for the operation of embodiments of the current inventive concept. The processing element may be in communication with the other electronic components through serial or parallel links that include address busses, data busses, control lines, and the like.

The memory elements 26 may include data storage components such as read-only memory (ROM), programmable ROM, erasable programmable ROM, random-access memory (RAM) such as static RAM (SRAM) or dynamic RAM (DRAM), cache memory, hard disks, floppy disks, optical disks, flash memory, thumb drives, USB ports, or the like, or combinations thereof. The memory elements may include, or may constitute, a “computer-readable medium”. The memory elements may store the instructions, code, code segments, software, firmware, programs, applications, apps, services, daemons, or the like that are executed by the processing elements. The memory elements may also store settings, data, documents, sound files, photographs, movies, images, databases, and the like.

The communication element 28 generally allows communication with external systems or devices. The communication elements may include signal or data transmitting and receiving circuits, such as antennas, amplifiers, filters, mixers, oscillators, digital signal processors (DSPs), and the like. The communication elements may establish communication wirelessly by utilizing RF signals and/or data that comply with communication standards such as cellular 2G, 3G, or 4G, IEEE 802.11 standard such as WiFi, IEEE 802.16 standard such as WiMAX, Bluetooth™, or combinations thereof. Alternatively, or in addition, the communication elements may establish communication through connectors or couplers that receive metal conductor wires or cables which are compatible with networking technologies such as ethernet. In certain embodiments, the communication element may also couple with optical fiber cables. The communication elements may be in communication with or electronically coupled to memory elements and/or processing element.

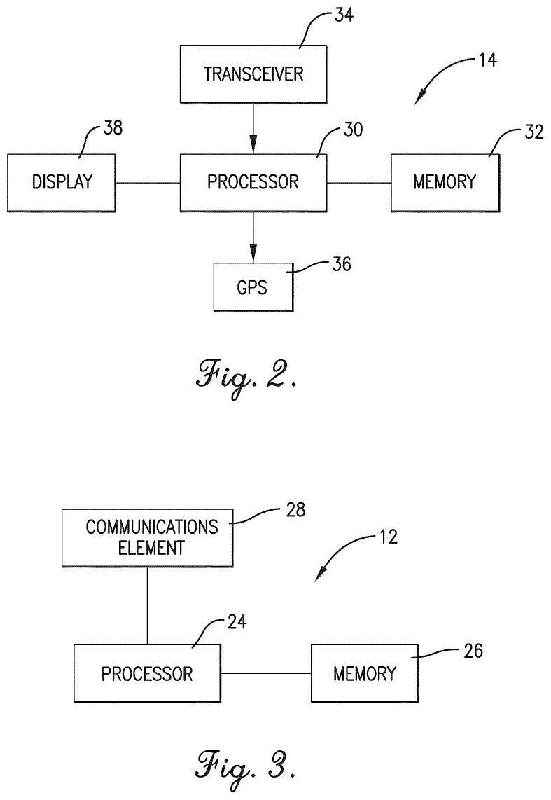

The personal computing devices 14, 16, 18 may be any devices used by patients and/or other users of the present invention. The personal computing devices, also referred to herein as “mobile computing devices”, are preferably mobile phones such as those provided by Apple but may also be smart watches, laptop computers, tablet computers, or similar electronic devices. As shown in FIG. 2 each of the devices 14, 16, 18 includes a processor or processing elements 30, memory or memory elements 32, a transceiver or other communications element 34, and an Internet browser that can access a conventional Internet connection such as a wireless broadband connection, DSL converter, or ISDN converter to exchange data with the computer server system 12 via the communications network 20. The personal computing devices 14, 16, 18 also include GPS receivers 36 and mapping capabilities to determine and display their current locations and locations of medical care centers as well as directions between them.

The mobile electronic devices also include displays 38 which may include video devices of the following types: plasma, light-emitting diode (LED), organic LED (OLED), Light Emitting Polymer (LEP) or Polymer LED (PLED), liquid crystal display (LCD), thin film transistor (TFT) LCD, LED side-lit or back-lit LCD, or the like, or combinations thereof. In various embodiments, the displays may also include a touch screen occupying the entire screen or a portion thereof so that the display functions as part of a user interface. The touch screen may allow the user to interact with the mobile electronic device by physically touching, swiping, or gesturing on areas of the screen.

The communications network 20 provides communications and data transfer between the server system 12; the personal computing devices 14, 16, 18; and/or other devices and may be the Internet or any other communications network such as a local area network, a wide area network, or an intranet. The communications network may include or be in communication with a wireless network 22 capable of supporting wireless communications such as the wireless networks operated by AT&T, Verizon, or T-Mobile. The wireless network 22 may include conventional switching and routing equipment. The communications network 20 and wireless network 22 may also be combined or implemented with several different networks.

The components of the system 10 illustrated and described herein are merely examples of computer equipment and software that may be used to implement embodiments of the present invention and may be replaced with other equipment without departing from the scope of the present invention. Some of the illustrated components of the system 10 may also be combined.

Embodiments of the present invention may also comprise one or more computer programs stored in or on computer-readable medium residing on or accessible by the computer server system 12 and/or the personal computing devices 14, 16, 18. The computer programs may comprise listings of executable instructions for implementing logical functions in the computers and can be embodied in any non-transitory computer-readable medium for use by or in connection with an instruction execution system, apparatus, or device, such as a computer-based system, processor-containing system, or other system that can fetch the instructions from the instruction execution system, apparatus, or device, and execute the instructions. In the context of this application, a “computer-readable medium” can be any non-transitory means that can contain, store, or communicate the programs. The computer-readable medium can be, for example, but not limited to, an electronic, magnetic, optical, electro-magnetic, infrared, or semi-conductor system, apparatus, or device. More specific, although not inclusive, examples of the computer-readable medium would include the following: an electrical connection having one or more wires, a portable computer diskette, a random access memory (RAM), a read-only memory (ROM), an erasable, programmable, read-only memory (EPROM or Flash memory), an optical fiber, and a portable compact disk read-only memory (CDROM).

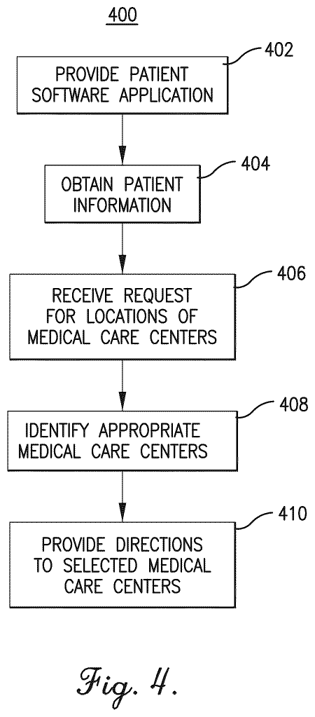

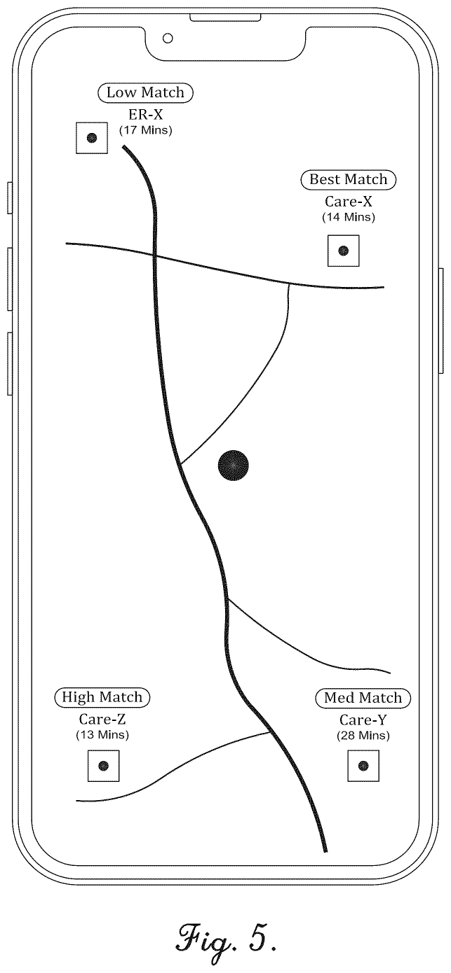

The flow chart of FIG. 4 shows the functionality and operation of a preferred implementation of the present invention in more detail. In this regard, some of the blocks of the flow chart may represent a computer-implemented method 400 and/or a module segment or portion of code of the computer programs of the present invention which comprises one or more executable instructions for implementing the specified logical function or functions. In some alternative implementations, the functions noted in the various blocks may occur out of the order depicted in FIG. 4. For example, two blocks shown in succession in FIG. 4 may in fact be executed substantially concurrently, or the blocks may sometimes be executed in the reverse order depending upon the functionality involved.

One embodiment of the invention is implemented with a patient software application that is executed on at least one of the mobile electronic devices 14, 16, 18. The patient software application may be downloaded from the server computer 12 or another source as depicted by block 402 in FIG. 4 and assists a patient in locating nearby medical care centers that provide appropriate medical care. In other embodiments of the invention, the steps and features described herein are performed by the portable electronic devices and the server computer without a patient software application.

In one embodiment, the patient software application gathers patient information from the user of the mobile electronic device that helps with locating appropriate medical care centers as depicted by block 404 in FIG. 4. For example, the patient software application may prompt a user/patient to enter their name, age, sex, and other personal information during their first use and/or registration of the application. The application may also prompt the user to identify any known prior medical issues, allergies, and family health conditions. Patient information may also be gathered by the server computer 12 or other computers from insurance records, doctor records, and other sources as permitted by healthcare laws and regulations. Additional patient information may also be gathered by the patient software application and/or the server computer as a patient uses the patient software application to locate medical care centers. For example, if a patient uses the application every year to find a medical care center that offers seasonal flu and/or COVID shots, relevant information about these prior uses may be stored for later analysis as described below. Patient information may also be gathered from wearable electronics such as fitness watches.

The patient software application is also configured to receive a request for locations of medical care centers near the patient as depicted by block 406 in FIG. 4. The request may be initiated by the patient by pressing a screen display button or icon or by entering text into a dialog box. The patient software application may employ or access natural language processing (NLP) so that requests and queries can be made and interpreted via everyday language. The patient software application may also prompt the patient for details about the requested medical care. For example, the patient software application may prompt the user to describe their current medical needs and/or rate the severity of their medical needs.

Once a request for locations of medical care centers is received by the patient software application, the application obtains the current location of the mobile electronics device on which the application is being executed and transmits data representative of the request, the location of the mobile electronic device, and any acquired patient information to the remote server 12.

The remote server 12 receives the data provided by the patient software application and analyzes it, along with other information, to provide recommendations for medical care centers to the patient software application as depicted by block 408 in FIG. 4. In one embodiment, the remote server determines locations of a number of medical care centers within a predetermined distance of the current patient location and sends information back to the patient software application that identifies these medical care centers as depicted by block 410 in FIG. 4. Mapping programs on or accessible by the related mobile electronic device may then provide driving directions and estimated driving times for each of the located medical care centers. The server computer may also employ, or access a source of, real-time traffic and route optimization to determine and suggest the fastest routes to medical care centers based on current conditions.

In another embodiment, the remote server 12 determines an estimated wait time for each of the identified medical care centers. Such wait times may be obtained directly from data provided by the medical care centers. When wait time data is not available from the medical care centers, wait times may be predicted by the remote server by analyzing historical wait times at the medical care centers, the current number of patients already checked in to the medical care centers, and other information. Such predicted wait times may be determined with artificial intelligence programs. The remote server then sends data representative of these wait times along with the locations of the medical care centers to the patient software application so the patient software application may display the locations, driving times, and wait times for the medical care centers.

In other embodiments, the remote server may analyze the above-described patient information and any details entered by the patient about the requested medical care to determine which of the nearby medical care centers are most appropriate for the patient. For example, if the patient described their medical needs as severe or urgent, the remote server may only identify and provide location information for hospital emergency rooms. Or, if the patient requests non-emergency care such as seasonal flu and/or COVID shots, the remote server may analyze the patient information to identify and provide location information for pharmacies or non-urgent care centers and/or medical care centers that the patient has visited in prior years for routine shots. The remote server and/or the patient software application may also employ an AI-powered chatbot that can conduct preliminary triage based on symptoms described by the patient and advise on the urgency of seeking medical care and to advise which of the nearby medical care centers are most appropriate for the patient.

In other embodiments, the server computer may implement AI capabilities to analyze patient feedback and ratings for medical care centers to identify and suggest only medical care centers that are highly recommended by other users.

FIG. 5 depicts an exemplary map that may be displayed on one of the portable electronic devices in response to a patient request for a nearby medical center. In this example, the patient requested a medical center for a COVID booster shot and a flu shot. The remote server analyzes the request along with other information described above and provides the patient software application information to be displayed for 4 nearby medical centers.

The first is a hospital emergency room and is identified by ER-X. ER-X has a combined drive and wait time of 17 minutes. But because the patient's current medical needs do not require a visit to a hospital emergency room, the remote server determines this emergency room is not the best choice of the 4 nearby medical care centers and directs the patient software application to indicate this medical care center as a “Low Match”.

The second medical care center is an urgent care center and is identified by Care-X. Care-X has a combined drive and wait time of 14 minutes. The patient information gathered by the patient software application and/or the remote server indicates the patient previously visited Care-X for a COVID booster. Based on the relatively short wait/drive time and the patient's prior experience with Care-X, the remote server determines Care-X is the best choice of the 4 nearby medical care centers and directs the patient software application to indicate this medical care center as a “Best Match”.

The third medical care center is an urgent care center and is identified by Care-Y. Although Care-Y is closer to the patient than Care-X, it has a longer wait time and therefore a longer combined drive and wait time of 28 minutes. Based on the relatively longer wait/drive time, the remote server determines Care-Y is a good choice but not best choice of the 4 nearby medical care centers and directs the patient software application to indicate this medical care center as a “Medium Match”.

The fourth medical care center is an urgent care center and is identified by Care-Z. Although Care-Z has a 13 minute combined wait/drive time, which is shorter than the wait/drive time for Care-X, the remote server determines Care-X is still the better choice based on the patient's prior experience with Care-X and directs the patient software application to indicate Care-Z as a “High Match” but not “Best Match”. Thus, in this example, the remoter server and patient software application encourages the patient to visit either Care-X or Care-Z.

The patient software application may also employ image recognition features for scanning and recognizing information from a patient's insurance card. The patient software application may also provide automated alerts for regular medical check-ups and preventive care.

### Additional Considerations

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments but is not necessarily included. Thus, the current technology can include a variety of combinations and/or integrations of the embodiments described herein.

Although the present application sets forth a detailed description of numerous different embodiments, the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as computer hardware that operates to perform certain operations as described herein.

In various embodiments, computer hardware, such as a processing element, may be implemented as special purpose or as general purpose. For example, the processing element may comprise dedicated circuitry or logic that is permanently configured, such as an application-specific integrated circuit (ASIC), or indefinitely configured, such as an FPGA, to perform certain operations. The processing element may also comprise programmable logic or circuitry (e.g., as encompassed within a general-purpose processor or other programmable processor) that is temporarily configured by software to perform certain operations. It will be appreciated that the decision to implement the processing element as special purpose, in dedicated and permanently configured circuitry, or as general purpose (e.g., configured by software) may be driven by cost and time considerations.

Accordingly, the term “processing element” or equivalents should be understood to encompass a tangible entity, be that an entity that is physically constructed, permanently configured (e.g., hardwired), or temporarily configured (e.g., programmed) to operate in a certain manner or to perform certain operations described herein. Considering embodiments in which the processing element is temporarily configured (e.g., programmed), each of the processing elements need not be configured or instantiated at any one instance in time. For example, where the processing element comprises, a general-purpose processor configured using software, the general-purpose processor may be configured as respective different processing elements at different times. Software may accordingly configure the processing element to constitute a hardware configuration at one instance of time and to constitute a different hardware configuration at a different instance of time.

Computer hardware components, such as communication elements, memory elements, processing elements, and the like, may provide information to, and receive information from, other computer hardware components. Accordingly, the described computer hardware components may be regarded as being communicatively coupled. Where multiple of such computer hardware components exist contemporaneously, communications may be achieved through signal transmission (e.g., over appropriate circuits and buses) that connect the computer hardware components. In embodiments in which multiple computer hardware components are configured or instantiated at different times, communications between such computer hardware components may be achieved, for example, through the storage and retrieval of information in memory structures to which the multiple computer hardware components have access. For example, one computer hardware component may perform an operation and store the output of that operation in a memory device to which it is communicatively coupled. A further computer hardware component may then, later, access the memory device to retrieve and process the stored output. Computer hardware components may also initiate communications with input or output devices, and may operate on a resource (e.g., a collection of information).

The various operations of example methods described herein may be performed, at least partially, by one or more processing elements that are temporarily configured (e.g., by software) or permanently configured to perform the relevant operations. Whether temporarily or permanently configured, such processing elements may constitute processing element-implemented modules that operate to perform one or more operations or functions. The modules referred to herein may, in some example embodiments, comprise processing element-implemented modules.

Similarly, the methods or routines described herein may be at least partially processing element implemented. For example, at least some of the operations of a method may be performed by one or more processing elements or processing element-implemented hardware modules. The performance of certain of the operations may be distributed among the one or more processing elements, not only residing within a single machine, but deployed across a number of machines. In some example embodiments, the processing elements may be located in a single location (e.g., within a home environment, an office environment or as a server farm), while in other embodiments the processing elements may be distributed across a number of locations.

Unless specifically stated otherwise, discussions herein using words such as “processing,” “computing,” “calculating,” “determining,” “presenting,” “displaying,” or the like may refer to actions or processes of a machine (e.g., a computer with a processing element and other computer hardware components) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The patent claims at the end of this patent application are not intended to be construed under 35 U.S.C. § 112(f) unless traditional means-plus-function language is expressly recited, such as “means for” or “step for” language being explicitly recited in the claim(s).

Although the invention has been described with reference to the embodiments illustrated in the attached drawing figures, it is noted that equivalents may be employed, and substitutions made herein without departing from the scope of the invention as recited in the claims.

Having thus described various embodiments of the invention, what is claimed as new and desired to be protected by Letters Patent includes the following:

1. A computer-implemented method for locating a medical care center for a patient, the computer-implemented method comprising:
providing a patient software application for execution on a mobile electronic device operated by the patient, the patient software application being configured to obtain patient location information, receive a request for locations of medical care centers near the patient, and transmit data representative of the patient location information and the request for locations of medical care centers near the patient;
receiving at a remote server the data representative of the patient location information and the request for locations of medical care centers near the patient;
determining, via the remote server, locations of a plurality of medical care centers within a predetermined distance of the patient location;
analyzing, via the remote server, data representative of an estimated wait time for each of the medical care centers within the predetermined distance of the patient location;
determining, via the remote server, a combined drive and wait time for each of the medical care centers based on the patient location, the locations of the medical care centers, and the estimated wait time for the medical care centers;
transmitting, from the remote server to the mobile electronic device operated by the patient, information representative of the combined drive and wait time for at least one of the medical care centers.

2. The computer-implemented method of claim 1, further comprising: analyzing, via the remote server, patient information; determining from the patient information which of the medical care centers are most relevant to the patient; and transmitting, from the remote server to the mobile electronic device operated by the patient, indications of which of the medical care centers are most relevant to the patient.

3. The computer-implemented method of claim 2, wherein the patient information is received from a patient profile created at least partially from information obtained from the patient.

4. The computer-implemented method of claim 2, wherein the patient information is entered by the patient into the patient software application.

5. The computer-implemented method of claim 2, wherein the patient information is derived at least partially from previous requests for locations of medical care centers sent by the patient.

6. The computer-implemented method of claim 1, wherein the data representative of an estimated wait time for each of the medical care centers within the predetermined distance of the patient location is obtained directly from the medical care centers.

7. The computer-implemented method of claim 1, wherein the data representative of an estimated wait time for each of the medical care centers within the predetermined distance of the patient location is determined by the remote server.

8. The computer-implemented method of claim 7, wherein the data representative of an estimated wait time for each of the medical care centers within the predetermined distance of the patient location is determined by the remote server based on historical wait times for the medical care centers and information representative of current patient check-ins at the medical care centers.

9. The computer-implemented method of claim 1, wherein the patient software application is configured to gather insurance information for the patient via image recognition.

10. The computer-implemented method of claim 9, wherein the data representative of an estimated wait time for each of the medical care centers within the predetermined distance of the patient location is determined by the remote server based partly on the insurance information for the patient.

11. The computer-implemented method of claim 1, further comprising receiving at the remote server real-time traffic data.

12. The computer-implemented method of claim 11, further comprising determining, via the remote server, the combined drive and wait time for each of the medical care centers based on the patient location, the locations of the medical care centers, the estimated wait time for the medical care centers, and the real-time traffic data.

13. A computer-implemented method for locating a medical care center for a patient, the computer-implemented method comprising:
providing a patient software application for execution on a mobile electronic device operated by the patient, the patient software application being configured to obtain patient location information, receive a request for locations of medical care centers near the patient, and transmit data representative of the patient location information and the request for locations of medical care centers near the patient;
receiving at a remote server the data representative of the patient location information and the request for locations of medical care centers near the patient;
determining, via the remote server, locations of a plurality of medical care centers within a predetermined distance of the patient location;
analyzing, via the remote server, data representative of an estimated wait time for each of the medical care centers within the predetermined distance of the patient location;
determining, via the remote server, a combined drive and wait time for each of the medical care centers based on the patient location, the locations of the medical urgent care centers, and the estimated wait time for the medical care centers; and
transmitting, from the remote server to the mobile electronic device operated by the patient, information representative of the combined drive and wait time for at least one of the medical care centers.

14. The computer-implemented method of claim 13, further comprising: analyzing, via the remote server, patient information; determining from the patient information which of the medical care centers are most relevant to the patient; and transmitting, from the remote server to the mobile electronic device operated by the patient, indications of which of the medical care centers are most relevant to the patient.

15. The computer-implemented method of claim 14, wherein the patient information is received from a patient profile created at least partially from information obtained from the patient.

16. The computer-implemented method of claim 14, wherein the patient information is entered by the patient into the patient software application.

17. The computer-implemented method of claim 14, wherein the patient information is derived at least partially from previous requests for locations of medical care centers sent by the patient.

18. The computer-implemented method of claim 13, wherein the data representative of an estimated wait time for each of the medical care centers within the predetermined distance of the patient location is obtained directly from the medical care centers.

19. The computer-implemented method of claim 13, wherein the data representative of an estimated wait time for each of the medical care centers within the predetermined distance of the patient location is determined by the remote server based on historical wait times for the medical care centers and information representative of current patient check-ins at the medical care centers.

20. The computer-implemented method of claim 1, further comprising receiving at the remote server real-time traffic data; and determining, via the remote server, the combined drive and wait time for each of the medical care centers based on the patient location, the locations of the medical care centers, the estimated wait time for the medical care centers, and the real-time traffic data.
